(19) United States
(12) Patent Application Publication
(10) Pub. No.: US 2022/0249781 A1
(43) Pub. Date: Aug. 11, 2022

(54) STATE DETECTOR ARRANGEMENT FOR STATE DETECTION OF A MEDICAMENT DELIVERY DEVICE

(71) Applicant: SHL MEDICAL AG, ZUG (CH)

(72) Inventor: Sheng-Wei Lin, Taoyuan City (TW)

(21) Appl. No.: 17/626,012

(22) PCT Filed: Jun. 19, 2020
(86) PCT No.: PCT/EP2020/067103

§ 371 Date: Jan. 10, 2022

(30) Foreign Application Priority Data
Jul. 16, 2019 (EP) 19186467.7

(51) Int. Cl. A61M 5/315; A61M 5/20

(52) U.S. Cl. CPC A61M 5/31568; A61M 5/20; A61M 2205/52; A61M 2205/3561; A61M 2205/3306

(57) ABSTRACT

A state detector arrangement for indicating a state of use of a medicament delivery device is presented, where the state detector arrangement has an electromagnetic wave detector, and a spring, a deformation of the spring providing an indication of the state of use of the medicament delivery device, wherein the electromagnetic wave detector is configured to detect electromagnetic waves that have propagated through the spring, wherein an intensity of the electromagnetic waves detected by the electromagnetic wave detector provides a measure of the deformation of the spring.

### TECHNICAL FIELD

The present disclosure generally relates to medicament delivery devices. In particular it relates to state detection of a medicament delivery device.

### BACKGROUND

Medicament administration by means of medicament delivery devices such as auto-injectors and auto-inhalers, is often nowadays carried out by the user. It is therefore desirable for the user to know in what state the medicament delivery device is when handling the medicament delivery device. The medicament delivery device may for example be in an initial state which is before medicament administration, or the medicament may have already been expelled.

WO2018/077880 discloses a state indicator and a drug delivery device. The state indicator comprises an outer housing structure designed to indicate a number of states of the drug delivery device. The state indicator is formed as a single indicator indicating a pre-injection state before injection, an injection state during injection, and post-injection state when the injection is completed. The state indicator may be formed as an optical indicator. The optical indicator may be provided by an electromagnetic radiation source configured to illuminate at least a part of a movable component such as a needle shroud or piston rod, at least one optical sensor unit configured to emit infrared radiation and adapted to detect infrared radiation reflected from the movable component, and at least one processing unit configured to determine a status value as a function of the measured reflected infrared radiation. The sensor may be a position or distance sensor determining the position and/or movement of the movable component.

### SUMMARY

An object of the present disclosure is to provide a state detector arrangement for a medicament delivery device which solves or at least mitigates problems of the prior art.

There is hence according to a first aspect of the present disclosure provided a state detector arrangement for indicating a state of use of a medicament delivery device, the state detector arrangement comprising: an electromagnetic wave detector, and a spring, a deformation of the spring providing an indication of the state of use of the medicament delivery device, wherein the electromagnetic wave detector is configured to detect electromagnetic waves that have propagated through the spring, wherein an intensity of the electromagnetic waves detected by the electromagnetic wave detector provides a measure of the deformation of the spring.

The state of extension of the spring, and hence the state of use of the medicament delivery device may thereby be determined.

The spring may for example be a helical or coil spring, or a spiral spring, or a clock spring, a torsion spring.

The deformation of the spring may provide a measure of a state of relax of the spring. The state of relax of the spring may be the state of extension of the helical spring.

One embodiment comprises processing circuitry, wherein the processing circuitry is configured to determine an intensity of the electromagnetic waves detected by electromagnetic wave detector, and wherein the processing circuitry is configured to determine the deformation of the spring based on the intensity of the detected electromagnetic waves.

According to one embodiment the processing circuitry is configured to compare the intensity with reference intensities to obtain a measure of the state of use of the medicament delivery device.

One embodiment comprises an electromagnetic wave emitting device, wherein the electromagnetic wave emitting device is configured to emit electromagnetic waves in a direction transverse to a direction of deformation of the spring through the spring.

According to one embodiment the electromagnetic wave emitting device is arranged externally relative to the spring and the electromagnetic wave detector is configured to detect electromagnetic waves inside the spring.

According to one embodiment the electromagnetic wave emitting device is arranged inside the spring and the electromagnetic wave detector is configured to detect electromagnetic waves outside the spring.

According to one embodiment the electromagnetic wave emitting device is a light source.

The light source may be configured to emit wavelengths in the infrared spectrum, the visible spectrum and/or the ultraviolet spectrum.

The electromagnetic waves propagating through the spring may according to one example be ambient light from the environment where the state detector arrangement is located.

According to one embodiment the light source may be a semiconductor light source such as a light emitting diode.

The light source could alternatively for example be an incandescent light source such as a traditional light bulb or a halogen light source.

According to one embodiment the electromagnetic wave detector is a photodetector.

One embodiment comprises a plunger rod, wherein the spring is configured to drive the plunger rod, wherein the deformation of the spring provides a measure of the position of the plunger rod and thereby the state of use of the medicament delivery device. In this case, the spring has dual functionality, i.e. to provide an indication of the state of use and to drive the plunger rod.

Alternatively, the spring may be a dedicated indicator spring. In this case, the only function of the spring is to provide an indication of the state of use of the medicament delivery device. The spring is in this case not configured to drive a plunger rod.

There is according to a second aspect of the present disclosure provided a medicament delivery device comprising a state detector arrangement according to the first aspect.

The medicament delivery device may for example be an auto-injector or an auto-inhaler.

One embodiment comprises a housing and a supplementary device configured to be attached to the housing, wherein the spring is provided in the housing and the electromagnetic wave detector forms part of the supplementary device.

According to one embodiment the electromagnetic wave emitting device forms part of the supplementary device.

According to one embodiment the housing has a portion which is at least partly transparent and/or is provided with through-openings, enabling the electromagnetic wave detector to detect light that has propagated through the spring.

By means of the through-openings ambient light may be emitted into the housing and through the spring. Alternatively, or additionally, the electromagnetic wave emitting device may be configured to emit light through the through-openings, from the supplementary device, into the housing and through the spring.

One embodiment comprises an indicator unit configured to display a state of use of the medicament delivery device.

Generally, all terms used in the claims are to be interpreted according to their ordinary meaning in the technical field, unless explicitly defined otherwise herein. All references to “a/an/the element, apparatus, component, means, etc. are to be interpreted openly as referring to at least one instance of the element, apparatus, component, means, etc.”, unless explicitly stated otherwise.

### BRIEF DESCRIPTION OF THE DRAWINGS

The specific embodiments of the inventive concept will now be described, by way of example, with reference to the accompanying drawings, in which:

FIG. 1 shows an example of a medicament delivery device;

FIG. 2 shows an exploded view of the medicament delivery device in FIG. 1;

FIG. 3 shows a block diagram of an example of a state detector arrangement;

FIG. 4 shows a longitudinal section of a distal end portion of the medicament delivery device in FIG. 1 in an initial state;

FIG. 5 shows a longitudinal section of a distal end portion of the medicament delivery device in a final state; and

FIG. 6 shows a longitudinal section of a medicament delivery device comprising a supplementary device.

### DETAILED DESCRIPTION

The inventive concept will now be described more fully hereinafter with reference to the accompanying drawings, in which exemplifying embodiments are shown. The inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Like numbers refer to like elements throughout the description.

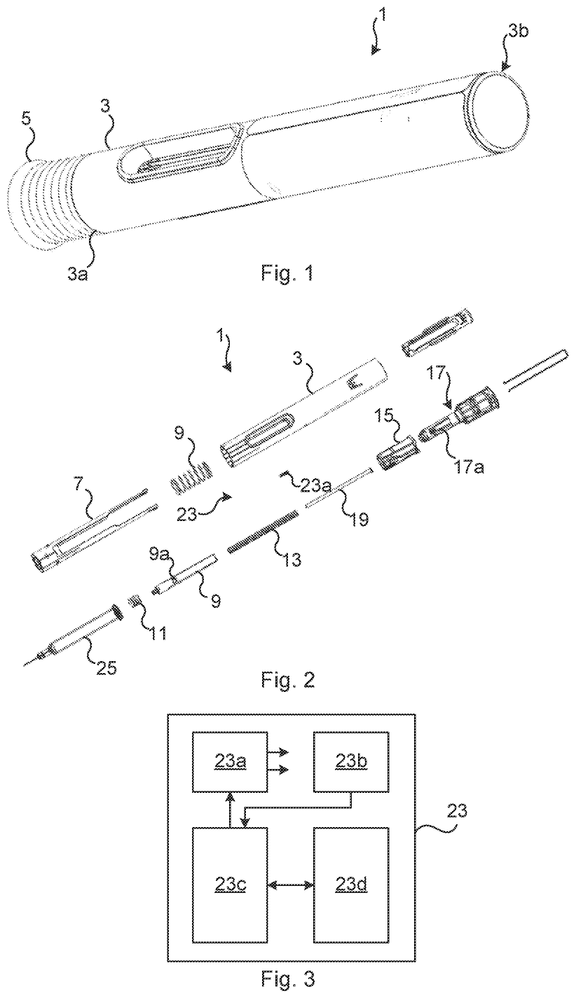

FIG. 1 shows an example of a medicament delivery device 1. The exemplified medicament delivery device 1 is an auto-injector but could alternatively for example be an auto-inhaler or an eye-dispenser.

The medicament delivery device 1 has a housing 3. The housing 3 has a proximal end 3a and a distal end 3b. The medicament delivery device 1 has a cap 5. The cap 5 is configured to be assembled with the housing 3.

The proximal end 3a of the housing 3 is configured to face the injection site during medicament administration. The distal end 3b is located opposite to the proximal end 3a. The proximal direction as used herein is along a central axis of the medicament delivery device 1 in a direction from the distal end 3b towards the proximal end 3a. The distal direction is the opposite direction relative to the proximal direction.

FIG. 2 depicts an exploded view of the medicament delivery device 1 showing some of its components. The medicament delivery device 1 comprises a delivery member cover 7, e.g. a needle cover, arranged to be received by the housing 3 and arranged to be biased in the proximal direction, a first energy accumulation member 9 arranged to bias the delivery member cover 7 in the proximal direction, a plunger rod 9 which is arranged to be biased proximally, a plunger 11, a spring 13, which can be a helical spring 13 in a preferred embodiment, arranged to bias the plunger rod 9 in the proximal direction, a tubular rotator 15 arranged to receive the plunger rod 9, a tubular extension part 17 which is axially and rotationally fixed relative to the housing 3, a rod 19 which the spring 13 is arranged to receive, and a state detector arrangement 23, of which an electromagnetic wave emitting device 23a is shown.

According to the present example, the plunger rod 9 has a radial opening 9a. The tubular extension part 17 which is arranged to receive the plunger rod 9 has a corresponding radial wing 17a flexible in the radial direction and arranged to engage with the radial opening 9a. The rotator 15 is arranged to receive a portion of the tubular extension part 17, in particular that portion which comprises the radial wing 17a. In its initial position the tubular rotator 15 is arranged to push the radial wing into engagement with the opening 9a preventing the plunger rod 9 from axial displacement.

The delivery member cover 7 has a distal end portion configured to interact with the rotator 15. The delivery member cover 7 is configured to slide linearly relative to the housing 3. By pushing the delivery member cover 7 into the housing 3, the delivery member cover 7 cooperates with the rotator 15, causing the rotator 15 to rotate. Linear movement of the delivery member cover 7 in the housing 3 is hence transformed into rotation of the rotator 15.

The rotator 15 has an inner surface provided with an inner structure that is recessed. When the rotator 15 is rotated, radial wings 17a will be moved into the recess(es) of the inner structure. The rotator 15 will thereby provide less radial force on the radial wing 17a, allowing the radial wing 17a to flex radially outwards to disengage from the plunger rod 9. The plunger rod 9, which is biased in the proximal direction by the spring 13, is thereby displaced axially and medicament administration is thus initiated as the plunger rod 9 pushes the plunger 11 into a medicament container 25.

FIG. 3 shows a block diagram of the state detector arrangement 23. The state detector arrangement 23 comprises the electromagnetic wave emitting device 23a and an electromagnetic wave detector 23b. The state detector arrangement 23 is configured to determine a state of extension/compression of the spring 13. The spring 13 is in this example a plunger rod spring. Since the compression and hence the longitudinal extension of the spring 13 determines the position of the plunger rod 9, the extension of the spring 13 also provides a measure of the state of use of the medicament delivery device 1.

The electromagnetic wave emitting device 23a may for example be a light emitting diode (LED). The electromagnetic wave detector 23b may for example be a photodetector.

The state detector arrangement 23 comprises processing circuitry 23c and a storage medium 23d. The storage medium 23d comprises computer code which when executed by the processing circuitry 23c enables the determination of the state of extension of the spring 13.

The storage medium 23d may for example be embodied as a memory, such as a random access memory (RAM), a read-only memory (ROM), an erasable programmable read-only memory (EPROM), or an electrically erasable programmable read-only memory (EEPROM) and more particularly as a non-volatile storage medium of a device in an external memory such as a USB (Universal Serial Bus) memory or a Flash memory, such as a compact Flash memory.

The processing circuitry 23c may use any combination of one or more of a suitable central processing unit (CPU), multiprocessor, microcontroller, digital signal processor (DSP), application specific integrated circuit (ASIC), field programmable gate arrays (FPGA) etc., capable of executing any herein disclosed operations concerning the determination of the state of extension of the spring 13.

The state detector arrangement 23 may furthermore comprise a battery configured to power the electromagnetic wave emitting device 23a, the processing circuitry 23c and the storage medium 23d. The battery may also be configured to power the electromagnetic wave detector 23b, depending on the implementation of the electromagnetic wave detector 23b.

The electromagnetic wave emitting device 23a is configured to irradiate the spring 13 from a direction transverse to the longitudinal extension of the spring 13 such that electromagnetic waves can propagate between the turns of the spring 13. The electromagnetic wave detector 23b is configured to detect electromagnetic waves that have propagated through the spring 13, i.e. between the turns of the spring 13. Since the spring 13 is a helical spring the amount of light that can pass between the turns of the spring depends on the state of compression of the spring 13.

The electromagnetic wave emitting device 23a may be arranged external to spring 13 and may be configured to emit electromagnetic waves from an external location through the spring 13. The electromagnetic wave detector 23b may in this case be arranged to detect the electromagnetic waves emitted by the electromagnetic wave emitting device 23a inside the spring 13. The electromagnetic wave detector 23b may for example be arranged aligned with the central axis of the spring 13, for example distally relative to a distal end of the spring 13.

According to one example the electromagnetic wave emitting device 23a may be arranged to emit electromagnetic waves from inside the spring 13 and the electromagnetic wave detector 23b may be configured to the detect electromagnetic waves at an external location relative to the spring 13. The electromagnetic wave emitting device 23a may for example be arranged aligned with the central axis of the spring 13, for example distally relative to a distal end of the spring 13.

According to one example the electromagnetic wave emitting device 23a may be arranged to emit electromagnetic waves from an external location through the spring 13. The electromagnetic wave detector 23b may be arranged to detect the electromagnetic waves emitted by the electromagnetic wave emitting device 23a externally to the spring 13. The electromagnetic wave detector 23b may for example be arranged opposite to the electromagnetic wave emitting device 23a, radially outside the spring 13.

One or more components of the medicament delivery device 1 may for example be made transparent or at least partly transparent for the wavelength(s) emitted by the electromagnetic wave emitting device 23a. For example, the tubular extension part 17 may be made of a material which is transparent or partly transparent for the wavelength(s) emitted by the electromagnetic wave emitting device 23a. The electromagnetic wave emitting device 23a may in this case be arranged to emit electromagnetic waves through the transparent or partly transparent portion of the tubular extension part 17 so that they propagate through the spring 13.

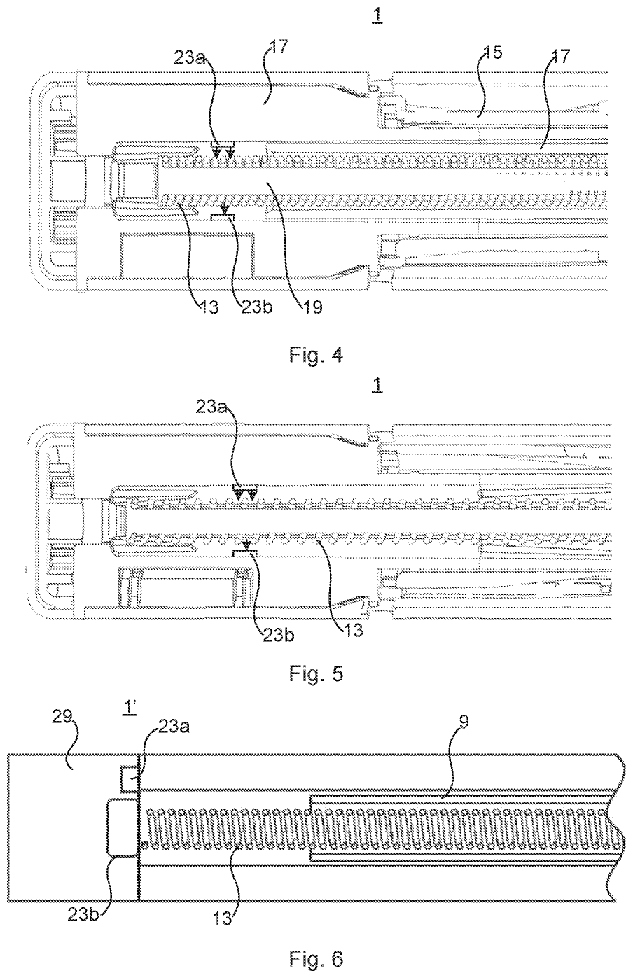

FIG. 4 shows a longitudinal section of a distal end portion of the medicament delivery device 1. The medicament delivery device 1 shown in FIG. 4 is in an initial state, prior to medicament administration.

The tubular extension part 17 is hollow and receives part of the plunger rod 9. The rod 19 is arranged inside the plunger rod 9, and the spring 13 is arranged around the rod 19. The spring 13 is arranged in a compressed state and biases the plunger rod 9 in the proximal direction. The radial wings 17a of the tubular extension part 17 engage with the corresponding openings 9a of the plunger rod 9. The plunger rod 9 is thereby maintained in its initial position.

In the present example, the electromagnetic wave emitting device 23a is arranged radially outside of the spring 13. The electromagnetic wave detector 23b is arranged radially outside of the spring 13. The electromagnetic wave detector 23b may for example be arranged opposite to the electromagnetic wave emitting device 23a. The electromagnetic wave detector 23b is arranged to detect light emitted by the electromagnetic wave emitting device 23a which has passed between the turns of the spring 13.

As electromagnetic waves emitted by the electromagnetic wave emitting device 23a pass through the spring 13, they are detected by the electromagnetic wave detector 23b. The electromagnetic wave detector 23b is configured to detect the intensity of the electromagnetic waves. This applies to any example disclosed herein. The processing circuitry 23c is configured to analyse the electromagnetic waves detected by the electromagnetic wave detector 23b. The processing circuitry 23c may be configured to determine an intensity of the electromagnetic waves detected by the electromagnetic wave detector 23b. The processing circuitry 23c may be configured to compare the intensity with reference intensities to obtain a measure of the state of extension of the spring 13. Each reference intensity may for example be associated with a specific state of the medicament delivery device 1. The state of use of the medicament delivery device 1 may thereby be determined. For example, when the intensity of the detected electromagnetic waves matches with a low intensity reference intensity, it can be determined that the medicament delivery device is in the initial state, as is the case in FIG. 4.

FIG. 5 shows the medicament delivery device 1 in a final state. The final state is obtained after a user has pushed the delivery member cover 7 in the distal direction into the housing 3, causing the rotator 15 to rotate and release the plunger rod 9. The plunger rod 9 is thereby moved in the proximal direction into the medicament container and medicament is expelled from the medicament delivery device 1. The final state is obtained when the plunger rod 9 has moved fully in the proximal direction.

The pitch or distance between subsequent turns of the coil structure of the spring 13 is increased as the compression of the spring 13 decreases. More electromagnetic waves can therefore pass through the spring 13. The intensity of the electromagnetic waves detected by the electromagnetic wave detector 23b is therefore increased. The processing circuitry 23c will in this case match the intensity of the electromagnetic waves with a higher intensity reference intensity than in the initial state of the medicament delivery device 1 illustrated in FIG. 4. The state of extension of the spring 13 may hence be determined. Consequently, the state of use of the medicament delivery device 1 may be determined.

The medicament delivery device 1 may comprise an indicator unit configured to indicate the state of use of the medicament delivery device 1. The indicator unit may for example be a display or a mechanical indicator. Alternatively, or additionally, the medicament delivery device 1 may be configured to transmit the state of use of the medicament delivery device 1 as determined by the processing circuitry 23a as data to an external device such as a computer, smart phone or tablet computer.

FIG. 6 shows another example of a medicament delivery device 1′. The medicament delivery device 1′ is similar to the medicament delivery device 1. Medicament delivery device 1′ is however configured to be used with a supplementary device 29. The medicament delivery device 1′ comprises the supplementary device 29. The supplementary device 29 may be configured to be mounted to the distal end 3b of the housing 3. The housing 3 and/or the end cap of the medicament delivery device 1′, comprises an interface configured to enable attachment of the supplementary device 29.

The supplementary device 29 may comprise the state detector arrangement 23, and a battery. The housing 3 and/or the interface may be at least partly transparent for a wavelength of the electromagnetic waves emitted by the electromagnetic wave emitting device 23a. The electromagnetic wave emitting device 23a is configured to emit electromagnetic waves through the housing and/or interface through the spring 13 in a direction transverse to the longitudinal extension of the spring 13.

The electromagnetic wave detector 23b is configured to detect electromagnetic waves that have passed between turns of the spring 13. The electromagnetic wave emitting device 23a may be configured to irradiate the spring 13 from a location radially outside of the spring 13 and the electromagnetic wave detector 23b may be configured to detect light that has passed between turns of the spring 13 to the inside of the spring 13. Alternatively, the electromagnetic wave emitting device 23a may be configured to irradiate the spring internally and the electromagnetic wave detector 23b may be configured to detect light that has passed through the spring from the inside of the spring and moved between turns of the spring 13 to the outside of the spring 13. According to one variation, electromagnetic wave emitting device 23a is configured to irradiate the spring 13 from an external location relative to the spring 13, and the electromagnetic wave detector 23b is configured to detect electromagnetic waves that have passed through the spring at a location which is radially outside of the spring 13, similar to the example shown in FIGS. 4-5.

The supplementary device 29 may be configured to indicate the state of use of the medicament delivery device 1′. For example, the supplementary device 29 may be provided with an indicator unit, such as a display, configured to display the state of use of the medicament delivery device 1′, as determined by the processing circuitry 23c. Alternatively or additionally, the supplementary device 29 may be configured to transmit the state of use of the medicament delivery device 1′ for example wirelessly, to a computer, smart phone, or tablet computer.

According to one example the housing may be provided with through-openings configured to allow ambient light to propagate into the housing, through the spring for detection by the electromagnetic wave detector.

The inventive concept has mainly been described above with reference to a few examples. However, as is readily appreciated by a person skilled in the art, other embodiments than the ones disclosed above are equally possible within the scope of the inventive concept, as defined by the appended claims.

1-13. (canceled)

14: A state detector arrangement for indicating a state of use of a medicament delivery device, the state detector arrangement comprising:
an electromagnetic wave detector, and
a spring, a deformation of the spring providing an indication of the state of use of the medicament delivery device,
wherein the electromagnetic wave detector is configured to detect electromagnetic waves that have propagated through the spring, wherein an intensity of the electromagnetic waves detected by the electromagnetic wave detector provides a measure of the deformation of the spring.

15: The state detector arrangement as claimed in claim 14, comprising processing circuitry, wherein the processing circuitry is configured to determine an intensity of the electromagnetic waves detected by electromagnetic wave detector, and wherein the processing circuitry is configured to determine the deformation of the spring based on the intensity of the detected electromagnetic waves.

16: The state detector arrangement as claimed in claim 15, wherein the processing circuitry is configured to compare the intensity with reference intensities to obtain a measure of the state of use of the medicament delivery device.

17: The state detector arrangement as claimed in claim 14, comprising an electromagnetic wave emitting device, wherein the electromagnetic wave emitting device is configured to emit electromagnetic waves in a direction transverse to a direction of deformation of the spring through the spring.

18: The state detector arrangement as claimed in claim 17, wherein the electromagnetic wave emitting device is arranged externally relative to the spring and the electromagnetic wave detector is configured to detect electromagnetic waves inside the spring.

19: The state detector arrangement as claimed in claim 17, wherein the electromagnetic wave emitting device is arranged inside the spring and the electromagnetic wave detector is configured to detect electromagnetic waves outside the spring.

20: The state detector arrangement as claimed in claim 17, wherein the electromagnetic wave emitting device is a light source.

21: The state detector arrangement as claimed in claim 20, wherein the light source is a light emitting diode.

22: The state detector arrangement as claimed in claim 14, wherein the electromagnetic wave detector is a photodetector.

23: The state detector arrangement as claimed in claim 14, comprising a plunger rod, wherein the spring is configured to drive the plunger rod, wherein deformation of the spring provides a measure of the position of the plunger rod and thereby the state of use of the medicament delivery device.

24: A medicament delivery device comprising a state detector arrangement as claimed in claim 14.

25: The medicament delivery device as claimed in claim 24, comprising a housing and a supplementary device configured to be attached to the housing, wherein the spring is provided in the housing and the electromagnetic wave detector forms part of the supplementary device.

26: The medicament delivery device as claimed in claim 24, wherein the housing has a portion which is at least partly transparent and/or is provided with through-openings, enabling the electromagnetic wave detector to detect light that has propagated through the spring.

27: The medicament delivery device as claimed in claim 24, comprising an indicator unit configured to display a state of use of the medicament delivery device.

28: A state detector arrangement for indicating a state of use of a medicament delivery device, the state detector arrangement comprising:
an electromagnetic wave detector;
a spring having a longitudinal axis, where a deformation along the longitudinal axis provides an indication of the state of use of the medicament delivery device; and
an electromagnetic wave emitting component that emits electromagnetic waves through the spring in a direction transverse to the deformation of the spring,
wherein the electromagnetic wave detector detects electromagnetic waves that have propagated through the spring, and
wherein an intensity of the electromagnetic waves detected by the electromagnetic wave detector provides a measure of the deformation of the spring.

29: The state detector arrangement as claimed in claim 27, wherein the electromagnetic wave detector is a photodetector and the electromagnetic wave emitting component is a light source.
